(19) United States
(12) Patent Application Publication
(10) Pub. No.: US 2023/0203374 A1
(43) Pub. Date: Jun. 29, 2023

(54) METHOD AND SYSTEM FOR PRODUCING A CARBONATE-CONTAINING SPECIES-RICH, NITROGEN-CONTAINING SPECIES-FREE SOLUTION

(71) Applicant: ECOLE POLYTECHNIQUE FEDERALE DE LAUSANNE (EPFL), Lausanne (CH)

(72) Inventors: Dimitrios TERZIS, St-Sulpice (CH); Lyesse LALOUI, Eclubens (CH)

(73) Assignee: ECOLE POLYTECHNIQUE FEDERALE DE LAUSANNE (EPFL), Lausanne (CH)

(21) Appl. No.: 17/925,960

(22) PCT Filed: May 19, 2020
(86) PCT No.: PCT/IB2020/054734

§ 371 Date: Nov. 17, 2022

(51) Int. Cl. C09K 17/10; C04B 12/00; C12M 1/04; C12M 1/00; C12N 9/80; E02D 3/12

(52) U.S. Cl. CPC C09K 17/10; C04B 12/007; C12M 23/24; C12M 23/58; C12M 29/04; C12N 9/80; C12Y 305/01005; E02D 3/12; C04B 2111/00732

(57) ABSTRACT

A method for producing a carbonate-containing species-rich, nitrogen-containing species-free solution from a urea-rich solution is proposed. The method comprising the steps of: providing a first reservoir comprising a first mixture including urea and a catalyser comprising an enzymatic catalyser and/or a microorganism; allowing an enzymatic reaction catalysed by the catalyser to decompose urea, thereby obtaining a second mixture comprising nitrogen-containing species and carbonate-containing species; converting at least some of the nitrogen-containing species into gaseous nitrogen-containing species to obtain a third mixture comprising the gaseous nitrogen-containing species and the carbonate-containing species; filtering the third mixture by a gas- permeable filter, thereby separating at least some of the gaseous nitrogen-containing species from the carbonate-containing species while keeping the catalyser away from the gas-permeable filter; and collecting the so-obtained carbonate-containing species-rich, nitrogen-containing species-free solution.

### TECHNICAL FIELD

The present invention generally belongs to the field of inorganic chemistry. More particularly, the invention pertains to a method and system for producing a carbonate-containing species-rich, nitrogen-containing species-free solution from a urea-rich solution, as well as applications for ground bio-consolidation processes.

### BACKGROUND OF THE INVENTION

Ground or soil consolidation solutions have been developed in the past 50 years to improve the properties of soils, foundations and to ensure the structural integrity of civil infrastructure under natural hazards, such as earthquakes, erosion and water level rise. Current solutions are limited to the use of cement, lime, petroleum-based chemicals (such as polyurethanes) and micro-silicates. These solutions, however, come at a heavy environmental cost since they lead to high alkalinity environments, with pH values often exceeding 12. Their application is complex, and these solutions often require the application of high pressures, which exceed 200 bars (20 MPa) in certain cases. Finally, petroleum-based chemicals result in microplastic pollution in the groundwater.

An alternative to the above solutions is reported in the past decade which mobilises soil microorganisms to produce calcium carbonate minerals. This application utilises ureolytic microbial strains which receive urea as intake and produce bicarbonate ions and ammonia cations. Aqueous ammonic species, i.e.

$${\text{NH}}_{4}^{+}$$

or the gaseous

$${\text{NH}}_{3}^{+}$$

pose a threat to the underground soil and water quality. According to the World Health Organization, acceptable limits of these species do not pass 0.2 mg/L. Values of ammonia reported in the literature reach 10,000 mg/L as part of the ureolytic-based carbonate mineralisation. A system to recycle ammonia (EP2804988A2) by flushing fresh water through a soil medium and pumping out the contaminated, ammonia-rich water, is not an optimal approach. This would require significant amounts of fresh water to dilute the ammonia from a 10,000 mg/L concentration to just 0.2 mg/L, and this would mean that the soil is saturated with water to enable a controlled flow field to ultimately guide the water towards an extraction well.

Another problem which emerges from the above approach is that ammonia, as a highly charged species, tends to adsorb on the negatively charged surface of soil particles (sand, silts, clays for example) and therefore its removal through flushing is not possible. Efforts have been reported through the use of zeolite filters, as proposed by Keykha et al., 2018, “Ammonium-Free Carbonate-Producing Bacteria as an Ecofriendly Soil Biostabilizer”, Geotechnical Testing Journal, 42(1), pp. 19-29. However, these solutions require complex maintenance of the mineral filter and cannot ensure large volume treatment in a fast and economic way. Other works suggest a down-flow hanging sponge (DHS) bioreactor system made of polyurethane sponges as proposed by Aoki et al., 2018, “A low-tech bioreactor system for the enrichment and production of ureolytic microbes”, Polish journal of microbiology, 67(1), pp.59-65, or by Omoregie et al., 2020, “A feasible scale-up production of Sporosarcina pasteurii using custom-built stirred tank reactor for in-situ soil biocementation”, Biocatalysis and Agricultural Biotechnology, p.101544. However, this latter system was originally developed for biofilm-type sewage treatment technology with biofilms representing highly colloidal species that can potentially clog the system’s capacity to filter liquid solutions.

### SUMMARY OF THE INVENTION

In order to address and overcome at least some of the above-mentioned drawbacks of the prior art solutions, the present invention proposes a reaction system and a related method having improved features and capabilities. More specifically, according to one aspect, the present invention aims to solve at least some of the problems identified above, and which are related to the use of bio-cement in geotechnical engineering applications to produce consolidated ground without residual chemicals in the soil.

In particular, a first purpose of the present invention is that of providing an easy and efficient process to exploit urea-rich solutions for producing carbonate-rich products suitable for ground consolidation purposes.

A further purpose of the present invention is that of providing a system and a method for efficiently separating carbonate-rich elements from nitrogen-containing elements and using the carbonate-rich elements and nitrogen-containing elements in separate processes.

Still a further purpose of the present invention is that of providing an all-in-one process for obtaining products suitable for ground consolidation purposes without the need of additional ground treatments.

All the above aims have been accomplished with the present invention, as described herein and in the appended claims.

In view of the above drawbacks of the prior art, according to the present invention there is provided a method for producing a carbonate-containing species-rich, nitrogen-containing species-free solution from a urea-rich solution according to claim 1.

The proposed method has the advantage that it allows an efficient separation of nitrogen-containing compounds, elements or species from carbonate-containing species (or carbonate-ions). The obtained carbonate-containing species-rich, nitrogen-containing species-free solution may then be used e.g. for ground or soil consolidation.

Thus, the proposed method may be used for carbonate bio-mineralisation of the ground while removing the harmful presence of ammonia. Therefore, no additional treatment is necessary to remove nitrogen species (which would in this case be contaminants in the ground) by washing the ground. By following the teachings of the present invention, it can be ensured that the contaminants do not enter the ground and they may be recycled and extracted in a prior step to be further valorised in other industrial systems. For example, the captured nitrogen-containing species can be used as a fertiliser or for the production of fertilisers (ammonia sulphate), or they may be used in chilled ammonia processes for capturing CO₂ from the air or fuel cells.

Another object of the present invention relates to a system for implementing the method of the invention according to claim 15.

Further features or variants of the present invention are defined in the appended claims.

### BRIEF DESCRIPTION OF THE DRAWINGS

Other features and advantages of the invention will become apparent from the following description of a non-limiting example embodiment, with reference to the appended drawings, in which:

FIG. 1 shows a simplified block diagram schematically illustrating an example reaction system according to the present invention; and

FIG. 2 shows a flow chart illustrating an example ground consolidation process of according to the present invention.

### DETAILED DESCRIPTION OF AN EMBODIMENT OF THE INVENTION

An embodiment of the present invention will now be described in detail with reference to the attached figures. Identical or corresponding functional and structural elements which appear in the different drawings are assigned the same reference numerals. It is to be noted that the use of words “first”, “second” and “third”, etc. may not imply any kind of particular order or hierarchy unless this is explicitly or implicitly made clear in the context. The embodiment explained in detail below relates to a new system and method for inducing consolidation of ground or soil. The proposed solution overcomes the problem of residual nitrogen-containing species, such as ammonium, in the ground when urea on the one hand, and bacteria (i.e. microorganisms) and/or enzymes on the other hand are used as reactive species to produce a calcified or consolidated soil.

It is to be noted that throughout the present description and claims, by a nitrogen-containing species-free or nitrogen species-free fluid or solution it is meant a fluid which is substantially free of nitrogen-containing species when compared with the initial number of nitrogen-containing species contained in the fluid to be filtered. Such species are, for example, ammonia, ammonium, ammonium chloride, nitrates, nitrites. Therefore, a substantially nitrogen-containing species-free fluid is considered to be a fluid which contains between 98% and 100% fewer nitrogen-containing species compared with the number of nitrogen-containing species before filtration. More preferably a nitrogen-containing species-free fluid contains between 99% and 99.9% less nitrogen compared with the amount of nitrogen before filtration. Stated otherwise, a nitrogen-containing species-free fluid is understood to contain between 0.0001 g/L and 0.0005 g/L of nitrogen-containing species. Thus, as long as the above constraints for the nitrogen-containing species are respected, the expression “nitrogen-containing species-free solution” or “nitrogen species-free solution” may be understood to mean a nitrogen-containing species-poor solution.

It is to be noted that throughout the present description and claims, by a carbonate-containing species-rich or carbonate species-rich fluid or solution it is meant a fluid which is substantially rich in carbonate-containing species, such as bicarbonate, carbonic acid or carbonate. More preferably a carbonate-containing species-rich fluid contains between 0.01 and 18 mol/L of carbonate-containing species, more preferably between 0.5 and 6 mol/L of carbonate-containing species. These species are produced following the breakdown of carbamide (urea) under the presence of enzymatic catalysers which are responsible for accelerating the breakdown. By a urea-rich solution is understood a fluid or solution which contains dissolved urea in concentrations between 0.01 mol/L and 18 mol/L and more preferably between 0.5 mol/L and 6 mol/L.

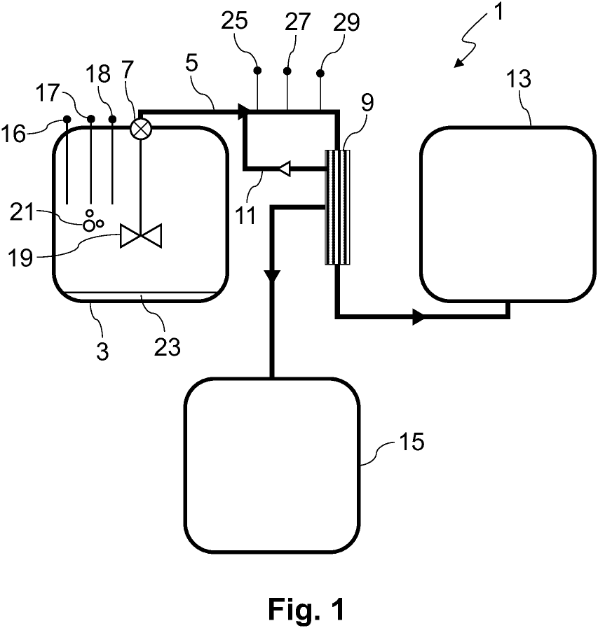

The block diagram of FIG. 1 schematically illustrates a reaction system 1 according to an example embodiment of the present invention. The system 1 comprises a first compartment 3, which in this example is a first chamber, container or reservoir, and more specifically a reaction chamber, where a bioreaction is arranged to take place as explained later in more detail. As shown in FIG. 1, the first chamber 3 is connected to a second compartment 5 via a pump or pump system 7, which is thus in this example located at an outlet of the first chamber. The second compartment is in this example a pipe or tube, and more specifically a longitudinal pipe, and optionally made of metal, having a cross section orthogonal to the pipe length axis (which is not necessarily a straight axis), in the range of 5 mm to 200 mm, and more specifically in the range of 10 mm to 50 mm. The second compartment 5 is connected to a third compartment 9, which comprises a first filtering element or unit (or simply a first filter), which in this example is a membrane filter comprising a plurality of fibres. The second compartment 5 can thus be understood to be a connection element operatively connecting the first chamber 3 and the membrane filter 9. A feedback connection element 11, which may be structurally substantially identical to the second compartment 5, is provided from the third compartment 9 to the second compartment 5 to selectively feed filtered mixture or solution back to the second compartment so that it can be filtered again by the membrane filter 9. It is to be noted that the system may comprise more than one pump, in particular two or three pumps. For example, one pump could be provided in the second compartment 5, and yet another pump in the feedback connection element 11. A fourth compartment 13, which in this example is a second chamber, container or reservoir, is also connected to the third compartment 9 through a pipe connection. The second chamber is arranged to receive and store nitrogen-containing filtering by-products, such as ammonia, ammonium sulphate, etc., which result from the filtering process taking place in or across the membrane filter. A fifth compartment 15, which in this example is a third chamber, container or reservoir, is also connected to the third compartment 9 through another pipe connection. The third chamber 15 is arranged to receive and store a carbonate-containing species-rich solution, which is the desired end solution that may be used to consolidate, reinforce, stabilise, strengthen, calcify, modify and/or improve the ground. The volume of all the chambers 3, 13, 15 would in typical applications be between 0.5 m³ and 20 m³.

The first chamber 3 is used to mix catalysers, such as urease enzymes and/or ureolytic microorganisms, with carbamide, also known as urea, in a starting or first mixture to decompose urea. This example thus uses urease, which is an enzyme that catalyses the hydrolysis of urea, to for example form ammonia and bicarbonate. More specifically, the first chamber 3 allows urea to be mixed with enzymes to allow breakdown of urea into nitrogen (N) and carbonate

$$\left({\text{CO}}_{\text{3}}^{\text{2-}}\right)$$

species to obtain a second mixture. Thus, the first chamber operates as a bioreactor. The first chamber may be configured so that is provides optimal conditions for the urea breakdown. For this purpose, in this example, the first chamber comprises a first operational conditions adjustment arrangement or system, which in this example comprises a first temperature controller 16, a first pH controller or stabiliser 17 and a first pressure controller 18 to respectively control the temperature, the pH, and the pressure inside the first chamber 3. It is to be noted that the first adjustment arrangement could instead comprise merely one or two of the above controllers. For example, the pressure controller 18 may be omitted in the first chamber. In this example, the temperature of the first chamber, and thus also the temperature of the liquid or solution inside the first chamber 3, is selected to be between 23° C. and 35° C., or more specifically between 28° C. and 32° C. The pH stabiliser 17 is used for stabilising the pH of the liquid in the first chamber 3. In this example, the pH is maintained between values 8 and 10, or more specifically between 9 and 9.5. Furthermore, the first pressure controller 18 is used to control the pressure of the liquid inside the first chamber in the case where the pressure controller happens to be present in the first chamber. In this example, the pressure of the liquid is selected to be between 0.01 Bars (1 kPa) and 3 Bars (300 kPa) or more specifically between 0.1 Bars (10 kPa) to 0.5 Bars (50 kPa).

In this example, the first chamber 3 further comprises a stirring device 19 or mixer, such as a mechanical mixer, to achieve homogenous mixing of the liquid in the first chamber, which is an aqueous solution comprising water, carbamide, and enzymatic compounds. The first chamber as shown in FIG. 1 further comprises an air supplier 21 to supply air into the first chamber 3 to accelerate the urea decomposition. A separation arrangement or separation means may also be provided in the first chamber to separate organic matter 23 from the liquid. The separation arrangement may be a second filter (not shown in the figures), which may be placed adjacent to the pump 7, for instance. Thanks to the separation arrangement, the second mixture, which is an ionic liquid, and which is free or substantially free of any organic matter, may be fed into the second compartment 5 by using the pump 7. The separation arrangement may, in addition, or alternatively, comprise the mixer 19. More specifically, the stirring operation of the mixer may be stopped for a given time duration to allow the organic matter to deposit at the bottom surface of the first chamber 3. The separation arrangement may, in addition, or alternatively be a centrifuge and/or a compound agglomeration arrangement. Thanks to the separation arrangement, only ionic liquid, which is substantially free of any organic matter, can enter into the second compartment 5. In other words, the first chamber 3 is able to retain enzymes in the first chamber and transfer only the nitrogen-rich and carbonate-rich liquid into the second compartment 5. When the first chamber 3 empties after pumping, it is filled with water and carbamide through an inlet (not shown in the drawings), and the residual enzymes are mixed and stirred again to continue executing the reaction.

The second compartment 5 comprises a second operational conditions adjustment arrangement or system, which in this example comprises a second temperature controller 25, a second pH controller or stabiliser 27 and a second pressure controller 29 to respectively control the temperature, the pH, and the pressure inside the second compartment 5. It is to be noted that the second adjustment arrangement could instead comprise merely one or two of the above controllers. The above controllers 25, 27, 29 are in this example configured to operate so that the operational conditions in the second compartment 5 are advantageously substantially the same as in the first chamber 3. In other words, the same parameter values are also valid in connection with the second operational conditions adjustment arrangement as mentioned above in connection with the first operational conditions adjustment arrangement. One or more of the above parameters are controlled to be able efficiently convert or transform the nitrogen-containing species in the ionic liquid into gaseous nitrogen-containing species to obtain a third mixture.

The obtained third mixture or solution is then arranged to be flushed through the membrane filter 9 where the gaseous nitrogen-containing species (i.e. ammonia gas, NH3) and the carbonate liquid phases are separated from each other. In the present example, the membrane filter 9 is made of polypropylene, and not of polyurethane for filtering the gas/liquid solution, and the membrane has a contact surface (i.e. the total surface in contact with the solution to be filtered) of at least 100 m² or more specifically at least 400 m². In this example, the membrane filter is a system of polymer hydrophobic fibres providing a large contact surface to permit liquid-gas exchanges. The membrane filter is thus a hydrophobic membrane letting only gases pass through it. The membrane has pores, in this case lamellar pores, with the greatest cross-sectional dimension of some micrometres or less, typically 0.3 µm (micrometres) to 10 µm, or more specifically between 0.4 µm and 1 µm, to allow gas to flow through it. Often, enzymes such as those used in the first chamber 3 are part of larger microbial cells which reach 2 µm in diameter and over 5 µm in length. Thus, the lamellar pore size is approximately five times smaller than the expected size of a single bacteria cell, which comprises the enzyme urease responsible for the breakdown of urea into carbonate and ammoniac. Further, such microbial cells represent colloidal substances that would attach onto fibres or other substrates hindering the gas-liquid exchanges. Typical filtration mechanisms would allow microbial attachment onto sponge or membrane networks to produce biofilms for remediation applications (Aoki et al., 2018, “A low-tech bioreactor system for the enrichment and production of ureolytic microbes”, Polish journal of microbiology, 67(1), pp.59-65). In the present invention, such attachment is unwanted and as it would hinder the proper functioning of the reactor system 1. This reaction system 1, independent of the catalytic reaction which takes place in the first chamber 3, allows for the separation of the nitrogen by-products without interrupting the catalytic chemical reaction in the first chamber, which produces such products.

Once the membrane filter 9 has separated the carbonate-containing species-rich liquid from the nitrogen-containing gas, then the nitrogen-containing species enter the second chamber 13. The extracted nitrogen-containing species may then be valorised in gas, liquid or solid form. Advantageously, the second chamber 13 is an acidic compartment rich in dissolved sulphates. This leads to the precipitation of ammonium sulphate in solid grains for extraction and/or collection. The carbonate-containing species-rich liquid is collected in the third chamber 15 for storage or direct injection into the ground.

By keeping the catalysers, such as microorganisms (which contain enzymes) and/or pure enzymes, in the first chamber 3, one can generate a continuous system which first degrades urea and then supplies the decomposed urea solution to the second compartment 5 for the filtering stage, through the pump 7. Expressed more broadly, the catalysers are kept away from the membrane filter. In other words, the catalysers are not allowed to come in contact with the membrane filter. By retaining the enzymes or catalysers in the first chamber, this process continues non-stop for an efficient and fast production of the nitrogen-carbonate liquid. Therefore, the present invention focuses on a reactor system which can greatly enhance the undisrupted preparation of a liquid solution and its proper filtering to provide nitrogen-free ground bio-consolidation. Extraction and recycling of the nitrogen by-products offers the advantage of a circular model for sustainable use of resources. The focus is put on filtering nitrogen species without degrading the filtering capacity of the membrane 9 due to enzyme or catalyser or microorganism migration from the first chamber 3. If the enzyme could escape the first chamber, ureolysis and therefore the production of nitrogen and carbonic species would be uncontrolled and therefore the level of extraction and recycling could not be controlled. The present invention therefore produces a known amount of nitrogen in the first chamber 3 and then extracts a known amount of nitrogen, for example at least 98% or 99.9% of the initial amount in the second compartment. In other words, of all the nitrogen species present in the second compartment 5, in this case 98% to 99.9% are filtered out by the filter membrane 9. If catalysers or organic matter reached the filter membrane 9, its filtering capacity would be reduced, which would mean that an unknown amount would not be recycled, and the filtered liquid cannot be introduced into the ground for the consolidation purposes as it would contain a significant amount of nitrogen gas, which could not be filtered by the clogged membrane filter.

Keeping the catalysers in the first chamber 3 allows for a continuous resupply of urea for increasing the production of nitrogen and carbonic species. If the system 1 allowed the migration of catalysers to the rest of the compartments, then few catalysers would remain in the first chamber to execute the urea breakdown. The present invention therefore proposes a single setup, which can operate in an undisruptive way to keep all the reactive species in their desired stage to in this manner avoid inter-compartment migration of species. By extracting the nitrogen species for reuse and the carbonic species for injection into the ground, a system to store these separated products can be implemented. This has the advantage of preparing the desired quantity and quality of liquids and storing them for final use or transport at the desired place of valorisation.

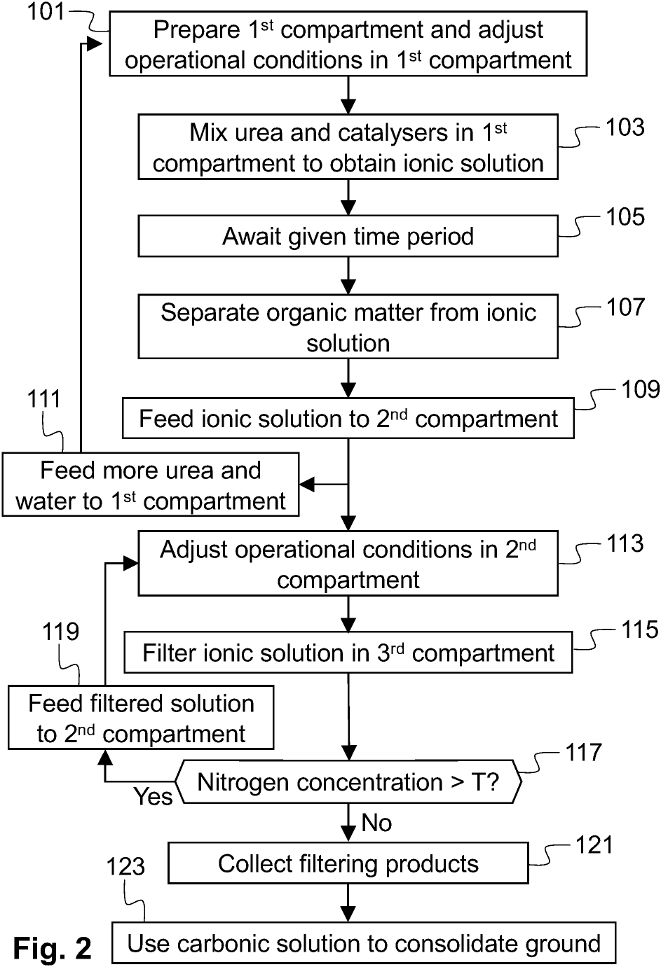

The flow chart of FIG. 2 presents an example method illustrating how the system of FIG. 1 may be used for ground bio-consolidation and/or for storing filtering products. In step 101, operational conditions are adjusted in the first chamber 3. This step may comprise adjusting the temperature and/or the pH, as well as optionally providing the correct amount of air into the first chamber. This step may also comprise providing the required mixing products, i.e. the first mixture into the first chamber. In step 103, urea and catalysers, which in this case are enzymes, are mixed in the first chamber. In step 105, a biochemical reaction is allowed to take place in the first chamber to obtain the second mixture. This process produces an ionic liquid solution comprising dissolved carbonate species

$${\text{CO}}_{\text{3}}^{\text{2-}} ,$$

as well as dissolved nitrogen species

$${\text{NH}}_{4}^{+} ,$$

and optionally also gaseous nitrogen compounds

$${\text{NH}}_{3}^{+}$$

the conditions in the first chamber permitting. It is to be noted that it may take hours or even days this bioreaction to fully take place. In step 107, the catalysers and any other organic matter are separated from the ionic liquid by using any one of the above-described separation means. In step 109, the ionic liquid or at least some of it, but substantially without the catalysers and the organic matter, is fed into the second compartment 5 by using the pump 7. Ideally, all the urea has now been broken down, and the first chamber 3 substantially emptied. In step 111, more mixing products, such as urea and water, and optionally also more catalysers, are brought into the first chamber 3 so that the mixing process can then continue by using the residual enzymes that have stayed in the first chamber, and optionally also the new enzymes. Thus, the process now continues in step 101 or 103 depending on whether or not the mixing conditions in the first chamber need to be readjusted. Parallel to step 111, in step 113 the conditions are adjusted in the second compartment 5 so that the environment becomes suitable for the production of gaseous nitrogen species. As a result of this operation, gaseous nitrogen species are produced in the second compartment. However, it is to be noted that instead of, or in addition to the production of the gaseous nitrogen species in the second compartment, they may be produced in the first chamber 3.

In step 115, the ionic liquid solution in the second compartment is fed or pumped, again by using the pump 7 to the third compartment 9 so that it can be filtered by the filter membrane. In step 117, it is determined whether or not the concentration of the nitrogen species, such as the aqueous

$${\text{NH}}_{4}^{+}$$

and the gaseous

$${\text{NH}}_{3}^{+} ,$$

in the filtered ionic liquid solution is above a given threshold T. More specifically, in this example, it is determined whether or not the filtered solution has the combined

$${\text{NH}}_{4}$$

and

$${\text{NH}}_{3}$$

concentrations above a given threshold, which may be for instance a percentage value between 0.1 and 5, or more specifically between 0.3 and 2, such as 0.5% (i.e. removal rate of 99.5%), of the initial concentration before the membrane filtering or a given weight/volume threshold value, such as any such value between 100 mg/L and 2000 mg/L, or more specifically any such value between 200 mg/L and 1000 mg/L, such as at least 400 mg/L. If the concentration of the nitrogen species is above the threshold, then in step 119, the filtered carbonic ionic liquid including residual nitrogen species is fed back to the second compartment 5 through the feedback connection element 11 so that it can be filtered again. This loop is then repeated until the nitrogen concentration drops below an acceptable value. If on the other hand, the concentration of the nitrogen species is not above the threshold, then in step 121, the nitrogen byproducts are collected in the second chamber 13 and the carbonic liquid solution, or the carbonate-containing species-rich, nitrogen-containing species-free solution, is collected in the third chamber 15. In step 123, the carbonate-containing species-rich, nitrogen-containing species-free solution is introduced, optionally directly, into the ground to consolidate the ground by CaCO₃ precipitation. This step may also involve providing a calcium source into the ground and allowing the formation of a cementitious product in the ground as a result of mixing the calcium source with the carbonate-containing species-rich, nitrogen-containing species-free solution without releasing ammonia or ammonium. The end product in the case of direct use of the carbonate-containing species-rich, nitrogen-containing species-free solution is calcium carbonate mineral products in the ground to consolidate and strengthen the ground. Alternatively, in step 123, one or both of the solutions from the second and third chambers 13, 15 may be stored for future use.

The above process as described with reference to the flow chart of FIG. 2 can be considered to be composed of two main operating steps: in a first step the production of a carbonate- and nitrogen-rich solution is obtained through biological fermentation and enzymatic catalysis; and in a second step this solution is purified to maximise the separation and valorisation of products. More precisely, the first chamber 3 utilises microorganisms and separates them from the carbonate- and nitrogen-rich solution to obtain a solution without organic matter. Subsequently the organic matter-free solution is adjusted for its temperature and/or pH and/or pressure and it is filtered by the membrane filter to separate liquid from gaseous phases. If organic matter is present in this step, then it would attach on the membrane and clog the nanopores, blocking the separation of gaseous and liquid phases. The third compartment 9 selectively removes and recycles gaseous nitrogen species from the carbonate-rich solution using membrane separation technology. The solution runs in a closed loop through the membrane allowing only gaseous species to pass through. The application of this system enables the preparation of a liquid solution which can enter the ground and consolidate it through the production of cementitious, calcite minerals while offering the possibility to recycle nitrogen for further valorisation.

The first, second, third, fourth and fifth compartments 3, 7, 9, 13, 15 act individually as reactive units and collectively as a system to separate products and recycle, store or use them directly. As described, the first chamber 3 can be used for successfully decomposing urea into nitrogen and carbonic species. However, the produced solution is not ready yet to be introduced into the ground and has to be treated for removing unwanted or hazardous by-products. The system 1, therefore, separates nitrogen species by means of filtering. However, if filtering occurs directly using the whole solution of the first compartment 3, which comprises the enzymes and the associated organic matter (enzymes), the whole filtering mechanism is put at risk due to clogging problems, accumulation of colloidal species on the filter surface and gradual loss of gas/liquid exchange capacity. Thus, the system could not sustain its purpose due to pore clogging and biofilm formation. Therefore, the proposed system of reactive and filtering processes is of unique value since it retains enzymatic and other organic species in the first compartment 3 and flows a liquid of already decomposed urea through the filter membrane. This avoids continuous and uncontrolled urea decomposition outside of the first compartment and ensures reduction of nitrogen species to the desired level for the final, ground consolidation liquid.

The manufacturing process of the proposed system 1 requires assembly of the pieces shown in FIG. 1, including the means or tools to achieve the extraction through pumping of the ionic liquid, and the means to adapt the temperature and pH. Some adjustments compared to existing tools are required to achieve the enzyme retention in the first compartment 3, such as a mechanical filtering or a retention time of few minutes for the enzymes to deposit at the compartment’s bottom surface via centrifuging for example. Therefore, the proposed system is easily assembled for industrial applications and ensures continuous monitoring of the reaction/extraction and collection processes.

The proposed system 1 serves multiple purposes, since except for the production of an ionic solution, which is external to the ground which can subsequently be consolidated, it can additionally achieve one or more of the following: (i) extract nitrogen species that should not remain as residuals in the ground or groundwater; (ii) recycle at least some part of the unwanted by-products and supply them to other industrial purposes; (iii) ensure undisrupted execution of the urea decomposition by retaining the enzymes in the first compartment; (iv) control the quality, condition and chemical composition of the liquid which will be injected into the ground; and (v) store the produced ionic species in tanks for future use. For instance, the nitrogen by-products can be removed from the system 1 and then be reused in either gas, liquid or solid form for future applications. The proposed system also minimises maintenance time and cost since no organic matter or enzymes or colloids enter the membrane filtering phase.

To summarise the above teachings, the present invention in the above-described embodiment proposes a system of compartments for bio-chemo-geological use, where the system combines preparation, separation, extraction and quality control of the produced bio-calcification liquid in a single setup. Contrary to prior art solutions, the present invention does not introduce unreacted urea into the ground and avoids the known and well-demonstrated detrimental effects of residual nitrogen on the quality of soil and groundwater. Urea fully or substantially fully reacts in the first chamber 3 and thus only carbonate is introduced into the ground. Furthermore, the same system serves as a platform of extracting by-products for efficient reuse through recycling in other industrial applications. Additionally, and optionally, to achieve efficient recycling, an optimal range of temperature, and pH is applied, as explained earlier, before the membrane filter 9 which leaves the carbonate-containing species-rich, nitrogen-containing species-free solution (i.e. the desired residual liquid solution) to optimal conditions for inducing soil consolidation. These conditions are optimal, if the temperature is above 25° C., and pH above 9 but below 9.5.

While the invention has been illustrated and described in detail in the drawings and foregoing description, such illustration and description are to be considered illustrative or exemplary and not restrictive, the invention being not limited to the disclosed embodiment. Other embodiments and variants are understood, and can be achieved by those skilled in the art when carrying out the claimed invention, based on a study of the drawings, the disclosure and the appended claims. Further variants may be obtained by combining the teachings of any of the features explained above.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A method for producing a carbonate-containing species-rich, nitrogen-containing species-free solution from a urea-rich solution, the method comprising the steps of:
providing a first reservoir comprising a first mixture including urea and a catalyser comprising an enzymatic catalyser and/or a microorganism;
allowing an enzymatic reaction catalysed by the catalyser to decompose urea, thereby obtaining a second mixture comprising nitrogen-containing species and carbonate-containing species;
converting at least some of the nitrogen-containing species into gaseous nitrogen-containing species to obtain a third mixture comprising the gaseous nitrogen-containing species and the carbonate-containing species;
filtering the third mixture by a gas-permeable filter, thereby separating at least some of the gaseous nitrogen-containing species from the carbonate-containing species while keeping the catalyser away from the gas-permeable filter ; and
collecting the so-obtained carbonate-containing species-rich, nitrogen-containing species-free solution.

2. The method according to claim 1, wherein the microorganism comprises a urease-producing microorganism.

3. The method according to claim 1, wherein the enzymatic catalyser comprises urease.

4. The method according to claim 1, wherein at least one of the first mixture, the second mixture and the third mixture is/are kept at a temperature above 20° C., preferably above 25° C., and wherein at least one of the first mixture, the second mixture and the third mixture is/are kept at a pH value comprised between 9 and 10.

5. The method according to claim 1, wherein the first mixture, the second mixture and the third mixture are aqueous mixtures.

6. The method according to claim 1, wherein the method further comprises separating the catalyser from the second mixture to keep the catalyser away from the gas-permeable filter.

7. The method according to claim 6, wherein the separation is carried out in the first reservoir.

8. The method according to claim 6, wherein the separation is carried out by at least one of the following means: a filtering device, a centrifuge, aggregation and gravity.

9. The method according to claim 1, wherein the method further comprises adjusting any one of temperature, pH, and pressure of the second mixture to convert at least some of the nitrogen-containing species into the gaseous nitrogen-containing species.

10. The method according to claim 9, wherein the conversion takes place in a connection element operatively connecting the first reservoir to the gas-permeable filter.

11. The method according to claim 1, wherein the method further comprises re-introducing at least some of the filtered third mixture back to the gas-permeable filter for further filtering.

12. The method according to claim 11, wherein, the method further comprises determining whether or not the concentration of the nitrogen-containing species in the filtered third mixture is above a given threshold value, and re-introducing at least some of the filtered third mixture back to the gas-permeable membrane for further filtering if the concentration of the nitrogen-containing species in the filtered third mixture is above the given threshold value.

13. The method according to claim 1, wherein the method further comprises collecting the separated gaseous nitrogen-containing species in a second reservoir, and the obtained carbonate-containing species-rich, nitrogen-containing species-free solution in a third reservoir.

14. A method for ground consolidation comprising the steps of:
performing the method according to claim 1, thereby producing the carbonate-containing species-rich, nitrogen-containing species-free solution;
flushing the carbonate-containing species-rich, nitrogen-containing species-free solution into the ground;
providing a calcium source into the ground; and
allowing the formation of a cementitious product in the ground as a result of mixing the calcium source with the carbonate-containing species-rich, nitrogen-containing species-free solution.

15. A system for producing a carbonate-containing species-rich, nitrogen-containing species-free solution from a urea-rich solution, the system comprising:
a first reservoir for a first mixture including urea and a catalyser comprising an enzymatic catalyser and/or a microorganism to allow an enzymatic reaction catalysed by the catalyser to decompose urea, thereby obtaining a second mixture comprising nitrogen-containing species and carbonate-containing species;
an operational conditions adjustment system for adjusting the operational conditions of the second mixture to allow at least some of the nitrogen-containing species to be converted into gaseous nitrogen-containing species to obtain a third mixture comprising the gaseous nitrogen-containing species and the carbonate-containing species;
a gas-permeable filter operatively connected with the first reservoir, and configured to separate at least some of the gaseous nitrogen-containing species from the carbonate-containing species to obtain a carbonate-containing species-rich, nitrogen-containing species-free solution while keeping the catalyser away from the gas-permeable filter;
a second reservoir configured to collect the separated gaseous nitrogen-containing species, and which is operatively connected with the gas-permeable filter; and
a third reservoir configured to collect the carbonate-containing species-rich, nitrogen-containing species-free solution, and which is operatively connected with the gas-permeable filter.

16. The system according to claim 15, wherein the third reservoir is configured to flush the carbonate-containing species-rich, nitrogen-containing species-free solution into the ground.

17. The system according to claim 15, wherein the operational conditions adjustment system comprises at least one of a temperature control system, and a pH control system, and a pressure control system, configured to control the temperature, pH, and pressure, respectively, inside the first reservoir and/or inside a connection element operatively connecting the first reservoir to the gas-permeable filter.

18. The system according to claim 15, wherein the gas-permeable filter has a contact surface of at least 100 m².

19. The system according to claim 15, wherein the gas-permeable filter is a liquid-impermeable filter.

20. The system according to claim 15, wherein the gas-permeable filter is a polypropylene fibrous membrane.

21. The system according to claim 15, wherein the gas-permeable filter is a porous element having a pore cross section between 0.3 µm to 10 µm, or more specifically between 0.4 µm and 1 µm.
